(19) United States
(12) Patent Application Publication
(10) Pub. No.: US 2024/0315940 A1
(43) Pub. Date: Sep. 26, 2024

(54) Topical Nourishing/Antimicrobial Compositions

(71) Applicant: Laboratory Skin Care, Inc., Hayward, CA (US)

(72) Inventor: Zahra Mansouri, Hayward, CA (US)

(21) Appl. No.: 18/693,708

(22) PCT Filed: Sep. 20, 2022
(86) PCT No.: PCT/US2022/044133

§ 371 Date: Mar. 20, 2024

Related U.S. Application Data
(60) Provisional application No. 63/246,572, filed on Sep. 21, 2021.

(51) Int. Cl. A61K 8/41; A61K 8/24; A61Q 17/00; A61Q 19/00; A61Q 19/10

(52) U.S. Cl. CPC A61K 8/416; A61K 8/24; A61Q 17/005; A61Q 19/007; A61Q 19/10

(57) ABSTRACT

Topical nourishing/antimicrobial compositions are provided. Embodiments of the compositions include an microbial agent, e.g., a quaternary ammonium compound, and calcium phosphate particles, where the compositions may optionally include one or more skin nourishing agents. Embodiments of the compositions include lotions and cleansers. Also provided are methods of making and using the compositions.

### CROSS-REFERENCE TO RELATED APPLICATIONS

Pursuant to 35 U.S.C. § 119 (e), this application claims priority to the filing date of U.S. Provisional Patent Application Ser. No. 63/246,572 filed Sep. 21, 2021; the disclosure of which application is herein incorporated by reference.

### INTRODUCTION

Skin is the body's outer covering, which protects against heat and light, injury, and infection. Skin regulates body temperature and stores water, fat, and vitamin D. The skin, which weighs about 6 pounds, is the body's largest organ. It is made up of two main layers: the epidermis or a surface layer, and a deeper connective tissue layer, known as the dermis. The epidermis undergoes continuous turnover as the outermost cells are exfoliated and replaced by cells that arise from inner dermal layers. The dermis is composed of a variety of cell types, including fibroblasts.

Excessive drying of the skin is a common problem which is often the result of exposure to wind, sun and low humidity, or a combination of these factors. Frequent washing of the hands can also result in excessive drying. This is particularly true if abrasive soaps, alcohol-based products and other harsh chemicals are used for cleansing.

Skin that has been excessively dried is not only unsightly, but also tends to slough off excessively and to crack, leading to abrasions of the skin surface. Because the skin serves a key role as a physical barrier to the entry of parasites and pathogens, excessive drying can lead to a breach of the barrier and infection by pathogenic bacteria and fungi. Thus, cracks or openings in the skin serve as a portal of entry for pathogens and potential pathogens. Even organisms that are normally considered to be non-pathogens can result in opportunistic infection in immunologically compromised individuals. Infections may be mild or severe and may be localized to the initial site(s) of infection or may be systemic and spread throughout the body. Such spread may occur by direct extension to contiguous tissues, or by way of the lymphatics and ultimately by way of the bloodstream.

Today, one of the greatest threats to humankind is the negative impact of contact cross-contamination. About 80% of infectious diseases are transmitted by touch and Millions of people die every year from infectious diseases (Centers for Disease Control and Prevention (CDC). CDC Quick Reference Guide for Public Information on Infection Control). Alcohol-based sanitizers are only effective until the alcohol evaporates (minutes). In addition, they can strip skin of healthy natural oils, moisture and radiance, damage skin's natural barrier function, can cause dermatitis, leave skin dry, cracked and vulnerable to bacterial and viruses and can be toxic to one's health and hazardous to the environment.

### SUMMARY

Topical nourishing/antimicrobial compositions are provided. Embodiments of the compositions include an antimicrobial agent, e.g., a quaternary ammonium compound, and calcium phosphate particles, where the compositions may optionally include one or more skin nourishing agents. Embodiments of the compositions include lotions and cleansers which are effective yet free of disinfectant/antiseptic alcohols, such as isopropyl alcohol and ethyl alcohol. Also provided are methods of making and using the compositions.

Aspects of the invention provide topical formulations with unexpected results which use a calcium phosphate component as a delivery system to bind with and deliver the benefits of calcium and skin nourishing agents, e.g., botanical and herbal extracts, anti-inflammatoires, anti-oxidants, vitamins and emollients, of the formulation into the skin where they work from inside out, while sanitizing active antimicrobial agent, e.g., benzalkonium chloride, remains on the skin's surface and kills 99.9% of microorganisms (Bacteria, Fungi & Viruses) and provides several hours (1-24, and 2-15, and 3-10, and 4-8 hours) of sanitizing protection while moisturizing and nourishing the skin.

### DEFINITIONS

“Admixture” or “blend” as generally used herein means a physical combination of two or more different components

By “contacting” is meant an instance of applying a composition to a contaminated surface.

“Contamination” is used herein to describe microbiological intrusions, such as the presence of toxins or pathogens, e.g., bacterial, fungi or viruses, in or on the surface of any material.

“Controlled release” as used herein means the use of a material to regulate the release of another substance.

“Effective amount” as used herein means an amount of a composition as disclosed herein, effective at dosages and for periods necessary to achieve the desired result.

“Environmentally friendly” as used herein refers to green, organic or natural compositions that are minimally harmful to the environment.

“Excipient” is used herein to include any other compound that may be contained in or on the microparticle that is not a therapeutically or biologically active compound. As such, an excipient should be pharmaceutically or biologically acceptable or relevant (for example, an excipient should generally be non-toxic to the subject). “Excipient” includes a single such compound and is also intended to include a plurality of excipients.

“Microbiological” as used herein refers to any inclusion or growth of harmful microorganisms such as mold, mildew, viral or bacterial contamination.

“Microbial Count” as used herein refers to the amount or number of microbiological contaminates present on any surface.

“Optional” or “optionally” means that the subsequently described event or circumstance can or cannot occur, and that the description includes instances where the event or circumstance occurs and instances where it does not.

“Primary biocide” is used herein to refer to compositions that are biologically active against microbial contaminates.

“Primary pathogen” is used herein to refer to mold, mildew, bacteria, fungi, viruses or other microorganisms that can cause contamination on a surface.

By “sufficient amount” and “sufficient time” means, an amount and time needed to achieve the desired result or results, e.g., control and/or prevention of microbial contamination.

“Surface” as used herein refers to the object that contains the microbiological contamination. The term surface can apply to the entire object, a portion or layer of the object, and down to the molecular structure of the object.

A “weight percent” of a component, unless specifically stated to the contrary, is based on the total weight of the formulation or composition in which the component is included.

### DETAILED DESCRIPTION

Topical nourishing/antimicrobial compositions are provided. Embodiments of the compositions include an antimicrobial agent, e.g., a quaternary ammonium compound, and calcium phosphate particles, where the compositions may optionally include one or more skin nourishing agents. Embodiments of the compositions include lotions and cleansers. Also provided are methods of making and using the compositions.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Topical Formulations

As summarized above, aspects of the invention include topical compositions (i.e., formulations) that are configured for application to a topical site of a human subject. Topical compositions of the invention are for applications to sites such as a keratinized skin surface of a mammalian subject, such as a human subject. By keratinized skin surface is meant a skin location of a subject, i.e., a location of the external covering or integument of an animal body. Because the topical compositions of the invention are formulated for delivery to topical location, they are formulated so as to be physiologically compatible with the topical location for which they are formulated. Accordingly, when contacted with the target keratinized skin surface for which they are formulated, the topical compositions of certain embodiments do not cause substantial, if any, physiological responses (such as inflammation or irritation) that would render the use of the topical compositions unsuitable for topical application.

As summarized above, topical compositions of the invention include: (a) an antimicrobial agent, e.g., a quaternary ammonium compound; (b) a calcium phosphate component, e.g., calcium phosphate particles, e.g., calcium phosphate particles such as porous calcium phosphate particles, which may be hydroxyapatite particles; and (c) a topical delivery vehicle, e.g., that includes one or more emollients, which delivery vehicle may vary depending on the nature of the topical composition, e.g., whether it is lotion or cleanser.

### Antimicrobial Agent

Topical compositions of the invention include an antimicrobial agent. Antimicrobial agents of interest include quaternary ammonium compounds, chlorhexidine, chloroxylenol, Cloflucarban, Fluorosalan, Hexachlorophene, Hexylresorcinol, Iodophors (Iodine-containing ingredients), Iodine complex (ammonium ether sulfate and polyoxyethylene sorbitan monolaurate), Iodine complex (phosphate ester of alkylaryloxy polyethylene glycol), Nonylphenoxypoly (ethyleneoxy) ethanoliodine, Poloxamer-iodine complex, Povidone-iodine, Undecoylium chloride iodine complex, Methylbenzethonium chloride, Phenol4, Secondary amyltricresols, Sodium oxychlorosene, Tribromsalan, Triclocarban, and the like. In some instances, the antimicrobial agent is not triclosan. In some instances, topical compositions of the invention include quaternary ammonium compounds. Quaternary ammonium compounds that may be present in compositions of embodiments of the invention include, but are not limited to: (C₁₂-C₁₄ alkyl)(C₁-C₂ dialkyl)benzyl ammonium salts, N-(C₁₂-C₁₈ alkyl)heteroaryl ammonium salts, and N-[(C₁₂-C₁₄ alkyl) (C₁-C₂ dialkyl)]heteroarylalkylene ammonium salts. Non-limiting examples of the (C₁₂-C₁₄ alkyl) (C₁-C₂ dialkyl)benzyl ammonium salts include (C₁₂-C₁₄ alkyl)dimethyl-benzyl ammonium chloride, (C₁₂-C₁₄ alkyl)dimethylbenzyl ammonium bromide, and (C₁₂-C₁₄ alkyl)dimethylbenzyl ammonium hydrogen sulfate. Non-limiting examples of the N-(C₁₂-C₁₈ alkyl)heteroaryl ammonium salts include cetyl pyridinium chloride, cetyl pyridinium bromide, and cetyl pyridinium hydrogen sulfide. For the N-(C₁₂-C₁₈ alkyl)heteroaryl ammonium salts other anions can be used.

Further examples of quaternary ammonium compounds suitable for use compositions of the invention include cetyltrimethylammonium chloride, stearyltrimethylammonium chloride, isostearyltrimethylammonium chloride, lauryltrimethylammonium chloride, behenyltrimethyl-ammonium chloride, octadecyltrimethylammonium chloride, cocoyltriinethylammonium chloride, cetyltrimethylammonium bromide, stearyltrimethylammonium bromide, lauryl-trimethylammonium bromide, isostearyllauryldimethylammonium chloride, dicetyldimethyl-ammonium chloride, distearyldimethylammonium chloride, dicocoyldimethylammonium chloride, gluconamidopropyldimethylhydroxyethylammonium chloride, di[polyoxyethylene(2)]oleylmethylammonium chloride, dodecyldimethylethylammonium chloride, octyldihydroxyethylmethylammonium chloride, tri[polyoxyethylene(5)]-stearylammonium chloride, polyoxypropylenemethyldiethylammonium chloride, lauryl-dimethyl(ethylbenzyl)ammonium chloride, behenamidopropyl-N,N-dimethyl-N-(2,3-dihydroxypropyl)ammonium chloride, tallowdimethylammoniopropyltrimethylammonium dichloride, benzethonium chloride (i.e., hyamine), and benzalkonium chloride.

In some instances, the quaternary ammonium compound is benzalkonium chloride, also known as alkyldimethylbenzylammonium chloride and by the trade name Zephiran (as well as N-Alkyl-N-benzyl-N,N-dimethylammonium chloride; ADBAC; BC50 BC80). Benalzalkonium chloride has the structure:

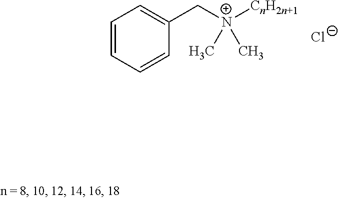

The amount of quaternary ammonium compound present in the compositions may vary, and in some instances the quaternary ammonium compound is present in an amount of from 0.001-5% by weight, such as from 0.05-2% by weight, and including from 0.1-1% by weight.

### Calcium Phosphate

Embodiments of the invention include a calicum phosphate component. The calcium phosphate component may vary, where calcium phosphate compounds that may make up the calcium phosphate component include, but are not limited to: tricalcium phosphate, dicalcium phosphate and its dihydrate, monocalcium phosphate and its monohydrate, hydroxyapatite, octacalcium phosphate, amorphous calcium phosphate, and the like, where in some instances the calcium phosphate component is hydroxyapatite.

In some instances, the calcium phosphate component is present as calcium phosphate particles. Particles present in embodiments of compositions of the invention may be porous calcium phosphate particles. By “porous” is meant that the particles have a porosity of 30% or more, such as 40% or more, including 50% or more, where the porosity may range from 30% to 85%, such as from 40% to 70%, including from 45% to 55%, as determined using a mercury intrusion porosimeter porosity determination protocol as described in ASTM D 4284-88 “Standard Test Method for Determining Pore Volume Distribution of Catalysts by Mercury Intrusion Porosimetry”. Porosity is also described by “pore volume (ml/g)” and in such instances many range from 0.1 ml/g to 2.0 ml/g. In some cases, the particles have a porosity such that their internal surface area ranges from 10 m²/g to 150 m²/g, such as from 20 m²/g to 100 m²/g, including 30 m2/g to 80 m²/g, as determined using a BET gas adsorption surface area determination protocol as described in ASTM D3663-03 Standard Test Method for Surface Area of Catalysts and Catalyst Carriers. The pore diameter may vary, ranging in certain instances from 2 to 100 nm, such as 5 to 80 nm, including 10 to 60 nm. In addition, the particles may have a tapping density ranging from 0.2 g/cm³ to 0.5 g/cm³, such as from 0.25 g/cm³ to 0.45 g/cm³, including from 0.3 g/cm³ to 0.4 g/cm³. The tap density can be measured by using standard ASTM WK13023-New Determination of Tap Density of Metallic Powders by a Constant Volume Measuring Method.

In some instances, the particles are rigid particles, where in some instances the rigid particles are uniform and spherical in shape. By “rigid” is meant that the particles are hard, such that they are not pliant. By “uniform” is meant that the shape of the particles does not vary substantially, such that the particles have substantially the same spherical shape. The term “spherical” is employed in its conventional sense to mean a round body whose surface is at all points substantially equidistant from the center. Of interest in certain embodiments are calcium particles having a diameter of 50 μm or less, such as 40 μm or less, 30 μm or less, 25 μm or less, e.g., 20 μm or less, such as 10 μm or less, including 5 μm or less, where in some instances the medium diameter is 4 μm or less, such as 3 μm or less, including 2 μm or less. Of interest in certain embodiments are calcium particulate compositions in which the median diameter of all of the particle members in the composition is 20 μm or less, such as 10 μm or less, including 5 82 m or less, where in some instances the medium diameter is 4 μm or less, such as 3 μm or less, including 2 μm or less. Of interest in certain embodiments are calcium phosphate particle compositions in which the arithmetic mean or average of all of the particles in the composition is 20 μm or less, such as 10 μm or less, including 5 μm or less, where in some instances the medium diameter is 4 μm or less, such as 3 μm or less, including 2 μm or less. With respect to the above ranges, in some instances the particles have a diameter of 0.01 μm or greater, e.g., 0.1 μm or greater, such as 0.5 μm or greater, including 1.0 μm or greater.

The particles are, in some instances, chemically pure. By chemically pure is meant that the particles are made up of substantially one type of compound, e.g., a calcium compound, such as a calcium phosphate mineral. Of interest as porous particles are calcium containing particles, such as calcium containing particles that are made of a molecule that includes calcium cation and a suitable anion, e.g., carbonate, phosphate, etc. In some instances, the particles are calcium carbonate particles, such as but not limited to the calcium carbonate particles disclosed in U.S. Pat. Nos. 5,292,495 and 7,754,176. In some instances, the calcium phosphate particles are made up of a calcium phosphate as exemplified by the molecular formula Ca₁₀(PO₄)₆(OH)₂.

In some instances, the particles are ceramic particles. By ceramic is meant that the particles are produced using a method which includes a step of subjecting the particles to high temperature conditions, where such conditions are illustrated below. High temperatures may range from 200 to 1000° C., such as 300 to 900° C. and including 300 to 800° C. In some embodiments, the particles have a compression rupture strength ranging from 20 to 200 MPa, such as from 50 to 150 MPa, and including 75 to 90 MPa, as determined using a SHIMADZU MCT-W500 micro-compression testing machine particle strength determination protocol with a particle sintered at a suitable temperature, e.g., 200° C. to 900° C., such as of 400° C. to 900° C., as described in European Patent EP1840661. In some embodiments, the particles are biodegradable, by which is meant that the particles degrade in some manner, e.g., dissolve, over time under physiological conditions. As the particles of these embodiments are bio-degradable under physiological conditions, they at least begin to dissolve at a detectable rate under conditions of pH of 5.5 or less, e.g., 5.3 or less, including 5 or less, e.g., 4.9 or less.

The uniform, rigid, spherical, porous calcium phosphate particles employed in embodiments of the invention may be prepared using any convenient protocol. In one protocol of interest, the particles are manufactured by spray drying a slurry which includes porous calcium phosphate (e.g., hydroxyapatite) crystals (which may range from 2 nm to 100 nm size range) to produce uniform spherical porous, e.g., nanoporous, calcium phosphate particles. The resultant particles are then sintered for a period of time sufficient to provide mechanically and chemically stable rigid spheres. In this step, the sintering temperatures may range from 100° C. to 1000° C., such as 200° C. to 1000° C., such as 300° C. to 900° C. and including 300° C. to 800° C. for a period of time ranging from 1 hour to 10 hours, such as 2 hours to 8 hours.

In some instances, the particles are loaded with one or more skin nourishing agents. Skin nourishing active agent-calcium phosphate particle complexes that are present in delivery compositions of these embodiments of the invention include calcium phosphate particles, such as porous calcium phosphate particles, e.g., nanoporous calcium phosphate particles, e.g., as described above, associated with one or more skin nourishing active agents. As the particles are associated with one or more skin nourishing active agents, one or more skin nourishing active agents are bound to the particles in some manner. The skin nourishing active agent(s) may be bound to the particles via a number of different associative formats, including but not limited to: ionic binding, covalent binding, Van der Waals interactions, hydrogen binding interactions, normal phase and reverse phase partition interactions, etc. As such, the particles may be described as being loaded with an amount of one or more skin nourishing active agents. By “loaded” is meant that the particles include an amount of one or more active agents (in other words an amount of a single active agent or two or more different active agents) that is together with, e.g., bound to or otherwise associated with, the particles. As the active agent is bound to the particles, the active agent does not dissociate from the particles in any substantial amount when the particles are present in the delivery composition. The amount of active agent component (which is made up of one or more distinct active agents) that is bound to the particles may vary depending on the particular active agent(s) making up the active agent bound particles, and in certain embodiments ranges from 0.01 to 1000 mg/g, such as from 0.1 to 750 mg/g and including 1 to 300 mg/g active agent(s)/gram particle.

When present, the active agent is reversibly associated with the calcium phosphate particles. By “reversibly associated” is meant that the active agent is released from the calcium phosphate particles following delivery to a subject, e.g., following application a delivery composition that includes the complexes to a skin site. As reviewed in greater detail below, the calcium phosphate particles of the complexes degrade under acidic conditions, such as under conditions of pH 5 or less, e.g., pH 4.9 or less, pH 4.7 or less, pH 4.5 or less, pH 4.3 or less. When the particles degrade, they release their active agent “payload”. The Stratum corneum (SC), the outer most layer of the skin, is made up roughly 20 layers of cells and is roughly 10 μm in thickness. The pH of the SC varies depending on its depth. Its outer most layers vary form pH 4.3 to 7.0, depending on the site sampled, or the individuals' sex. This pH rises to around 7.0 near the Stratum granulosum (SG). This rise is most dramatic in the last few layers of the SC adjoining the SG, as seen below. As such, as complexes of the invention penetrate into the SC, they degrade and concomitantly release any active agent associated therewith.

The released active agent retains its desired activity despite having been associated with the calcium phosphate particles in a complex. Accordingly, binding and release of the active agent to the calcium phosphate particles results in substantially little, if any, damage to the active agent. As such, the activity of the active agent is not diminished to an extent that adversely impacts its utility, where any reduction in activity caused by the association to the calcium phosphate particles that may occur with a given active agent is 25% or less, 10% or less, such as 5% or less and including 1% or less, e.g., as determined by an activity assay method.

In some embodiments, association of the active agent with the porous calcium phosphate particles in the complexes preserves one or more desirable features of the active agent, such as stability. In other words, the complex stabilizes the active agent, as compared to a control that lacks the calcium phosphate particles.

The topical compositions of the invention include an amount of the calcium phosphate particles, and optionally one or more skin nourishing active agents, present in a topical delivery vehicle. As reviewed above, in some instances the calcium phosphate particles are loaded with one or more skin nourishing active agents. In other instances, the one or more skin nourishing agents are otherwise present in the topical delivery vehicle. The amount of calcium phosphate particles that is present in the delivery composition and therefore combined with a delivery vehicle may vary. In certain embodiments the calcium phosphate particles are present in compositions in an amount ranging from about 0.001% or more by weight, such as 0.01%, or 0.05%, or 1% or more, 5% or more, 10% or more, 15% or more, 25% or more, 30% or more 50% or more. In certain embodiments, the calcium phosphate particles is added directly to the delivery vehicle (i.e., the calcium phosphate particles is not wetted prior to combining/mixing with the delivery vehicle). In other words, the calcium phosphate particles and the delivery vehicle are combined to form the topical composition.

### Topical Delivery Vehicle

The delivery vehicle (i.e., topical delivery component) refers to that portion of the topical composition that is not the calcium phosphate particles or quaternary ammonium compound. Delivery vehicles of interest include, but are not limited to, compositions that are suitable for applications via topical routes, e.g., lotions, soaps, and the like. In certain embodiments, the vehicle is formulated for application to a topical region or surface of a subject, such as a keratinized skin surface. The subject compositions may be formulated as stable solutions or suspensions of the components, e.g., in an aqueous solvent. Where desired, the components may be combined with one or more carrier materials to form a solution, suspension, gel, lotion, cream, ointment, aerosol spray, roll-on, foam products, mousses, or the like, as desired. Of interest in certain embodiments are aqueous delivery vehicles, i.e., aqueous vehicles that include a certain amount of water. Examples of aqueous vehicles include hydrogel vehicles, sprays, serums, etc.

The topical composition may also contain other physiologically acceptable excipients or other minor additives, particularly associated with organoleptic properties, such as fragrances, dyes, buffers, cooling agents (e.g., menthol), coating materials or the like. The excipients and minor additives will be present in conventional amounts, e.g., ranging from about 0.001% to 5%, such as 0.001-2%, by weight, and in some instances not exceeding a total of 10% by weight.

Lotions and cleansers (as well as other topical formulations) of interest may include one or more of the following components: Water, Viscosity modifiers, Humectants (e.g., propane diol)barb, Vegetable oils and hydrogenated vegetable oils, Emollients, Conditioning Agents, Emulsifiers, Glyceryl Esters of Fatty Acids, Silicone, C1-C30 monoesters and polyesters of sugar, Conditioning Agents, Preservatives, etc. Depending on the topical formulation, additional components of interest include: Abrasives, Absorbents, Antimicrobial and antifungal agents, Astringents, Anti-Acne agents, Anti-wrinkle agents, Anti-oxidants, Antimicrobials, Binders, Biological actives, Buffering actives, Bulking actives, Chelating agents, Chemical additives, External analgesics, Film former agents, Opacifying agents, pH adjusters, Reducing agents, Colorants, Fragrances, Cosmetic Soothing Agents, Tanning actives & accelerators, Skin lightening/whitening agents, Sunscreens, Surfactants, Skin Conditioning Agents, e.g., botanicals (such as but no limited to: Chamomilla Recutita (Matricaria) Flower Extract, Aloe Barbadensis Leaf Juice), Vitamins (e.g., Tocopherol acetate (vitamin E)), etc. As indicated above, of interest in certain embodiments are semi-solid delivery compositions, such as gels, creams and ointments. Such compositions may be mixtures of (in addition to the active agent) water, water soluble polymers, preservatives, alcohols, polyvalent alcohols, emulsifying agents, wax, solvents, thickeners (including but not limited to hydroxyethylcellulose), plasticizers, pH regulators, water-retaining agents and the like. Furthermore, such compositions may also contain other physiologically acceptable excipients or other minor additives, such as fragrances, dyes, buffers, coating materials or the like.

Emollients are compounds that replace or add to lipids and natural oils in the skin. The term emollient, as used herein, is intended to include conventional lipid materials (e.g., fats, waxes, and other water insoluble materials), polar lipids (e.g., lipid materials which have been modified to render them more water soluble), silicones and hydrocarbons. Emollients of interest include, but are not limited to: diisopropyl adipate, isopropyl myristate, isopropyl palmitate, ethylhexyl palmitate, isodecyl neopentanoate, C₁₂₋₁₅ alcohols benzoate, diethylhexyl maleate, PPG-14 butyl ether, PPG-2 myristyl ether propionate, Di-PPG-3 Myristyl Ether Adipate, cetyl ricinoleate, cholesterol stearate, cholesterol isosterate, cholesterol acetate, jojoba oil, cocoa butter, shea butter, lanolin, and lanolin esters.

Silicone oils may be divided into the volatile and non-volatile variety. The term “volatile” as used herein refers to those materials which have a measurable vapor pressure at ambient temperature. Volatile silicone oils of interest include but are not limited to: cyclic or linear polydimethylsiloxanes containing from 3 to 9, such as from 4 to 5, silicon atoms. Linear volatile silicone materials may have viscosities of 5 centistokes or less at 25° C., while cyclic materials may have viscosities of 10 centistokes or less. Nonvolatile silicone oils of interest include, but are not limited to: polyalkyl siloxanes, polyalkylaryl siloxanes and polyether siloxane copolymers. The essentially non-volatile polyalkyl siloxanes of interest include, for example, polydimethyl siloxanes with viscosities of 5 to 100,000 centistokes at 25° C.

Suitable esters include, but are not limited to: alkenyl or alkyl esters of fatty acids having 10 to 20 carbon atoms, such as isopropyl palmitate, isopropyl isostearate, isononyl isonanonoate, oleyl myristate, oleyl stearate, and oleyl oleate; ether-esters, such as fatty acid esters of ethoxylated fatty alcohols; polyhydric alcohol esters; ethylene glycol mono and di-fatty acid esters; diethylene glycol mono-and di-fatty acid esters, polyethylene glycol (100-6000) mono-and di-fatty acid esters; propylene glycol mono-and di-fatty acid esters, such as polypropylene glycol 2000 monooleate, polypropylene glycol 2000 monostearate, ethoxylated propylene glycol monostearate; glyceryl mono-and di-fatty acid esters; Glyceryl Stearate & PEG-100 Stearate; polyglycerol poly-fatty esters, such as ethoxylated glyceryl monostearate, 1,3-butylene glycol monostearate, 1,3-butylene glycol distearate; glycol distearate, polyoxyethylene polyol fatty acid ester; sorbitan fatty acid esters; and polyoxyethylene sorbitan fatty acid esters are satisfactory polyhydric alcohol esters; wax esters such as beeswax, spermaceti, myristyl myristate, stearyl stearate; sterols esters, of which soya sterol and cholesterol fatty acid esters are examples thereof. Both vegetable and animal sources of these compounds may be used. Examples of such oils include, but are not limited to: castor oil, lanolin oil, C10-18 triglycerides, caprylic/capric triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like. Also suitable are synthetic or semi-synthetic glyceryl esters, such as fatty acid mono-, di-, and triglycerides which are natural fats or oils that have been modified, for example, mono-, di-or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, and PEG glyceryl tallowates.

Fatty acids of interest include, but are not limited to: those having from 10 to 30 carbon atoms, such as pelargonic, lauric, myristic, palmitic, stearic, isostearic, hydroxystearic, oleic, linoleic, ricinoleic, arachidic, behenic and erucic acids.

Humectants of the polyhydric alcohol-type may also find use in the compositions, where examples of polyhydric alcohols include, but are not limited to: glycerol (also known as glycerin), polyalkylene glycols, alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. Also of interest are sugars, e.g., glucose, fructose, honey, hydrogenated honey, inositol, maltose, mannitol, maltitol, sorbitol, sucrose, xylitol, xylose, etc. When present, the amount of humectant may range from 0.001 to 25%, such as from about 0.005 to 20%, including from about 0.1 to 15%, where in some instances the amount of humectant ranges from 0.5 to 30%, such as between 1 and 15% by weight of the composition.

Emulsifiers may also be present in the vehicle compositions. When present, the total concentration of the emulsifier may range from 0.01 to 40%, such as from 1 to 20%, including from 1 to 5% by weight of the total composition. Emulsifiers of interest include, but are not limited to: anionic, nonionic, cationic and amphoteric actives. Nonionic surfactants of interest include those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono-and di-fatty acid esters of ethylene glycol; fatty acid monoglyceride; sorbitan, mono-and di-C₈-C₂₀ fatty acids; and polyoxyethylene sorbitan as well as combinations thereof. Alkyl polyglycosides and saccharide fatty amides (e.g., methyl gluconamides) are also of interest nonionic emulsifiers. Anionic emulsifiers of interest include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Where desired, preservatives can include in the compositions, e.g., to protect against the growth of potentially harmful microorganisms. Preservatives of interest include alkyl esters of para-hydroxybenzoic acid, hydantoin derivatives, propionate salts, and a variety of quaternary ammonium compounds. Specific preservatives of interest include, but are not limited to: iodopropynyl butyl carbamate, phenoxyethanol, methyl paraben, propyl paraben, imidazolidinyl urea, sodium dehydroacetate, benzyl alcohol, benzylhemiformal, benzylparaben, 5-bromo-5-nitro-1,3-dioxane, 2-bromo-2-nitropropane-1,3-diol, caprylyl glycol, ethylhexylglycerin, phenoxyethanol sorbic acid, methylparaben, propylparaben, ethylpareben, butylparaben, sodium benzoate, potassium sorbate, disodium salt of ethylenediaminetetraacetic acid, chloroxylenol, DMDM Hydantoin, 3-iodo-2-propylbutyl carbamate, chlorhexidine digluconate, phenoxyethanol, diazolidinyl urea, biguanide derivatives, calcium benzoate, calcium propionate, caprylyl glycol, biguanide derivatives, captan, chlorhexidine diacetate, chlorhexidine digluconate, chlorhexidine dihydrochloride, chloroacetamide, triclosan, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, Methylisothiazolinone/Methylchloroisothiazolinone, 1,2-Hexanediol & Caprylyl Glycol, and the like. When present, preservatives may be present in the delivery compositions in amounts ranging from about 0.01% to about 10% by weight of the composition. In some instances, the compositions are paraben free.

Thickening agents or viscosity modifiers may be included in the delivery compositions. Thickening agents of interest include, but are not limited to: polysaccharides, such as starches, natural/synthetic gums and cellulosics, etc. Starches of interest include, but are not limited to, chemically modified starches, such as aluminum starch octenylsuccinate. Gums of interest include, but are not limited to: xanthan, sclerotium, pectin, karaya, arabic, agar, guar (e.g., Cyamopsis Tetragonoloba (Guar) Gum), carrageenan, alginate and combinations thereof. Suitable cellulosics include, but are not limited to: hydroxypropyl cellulose, hydroxypropyl methylcellulose, hydroxyethylcellulose, ethylcellulose and sodium carboxy methylcellulose. Synthetic polymers are still a further class of effective thickening agent. This category includes crosslinked polyacrylates such as the Carbomers and polyacrylamides such as Sepigel® 305; Polyacrylamide & C13-14 Isoalkane & Laureth-7, etc. When present, amounts of the thickener may range from 0.001 to 5%, such as from 0.1 to 2%.

In some instances, natural or synthetic organic waxes may be present, e.g., one or more natural or synthetic waxes such as animal, vegetable, or mineral waxes. In some instances, such waxes will have a melting point ranging from 20 to 150° C., such as from 30 to 100° C., including 35 to 75° C. Examples of such waxes include waxes such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, and tribehenin. Also of interest are Acrocomia Aculeata Seed Butter, Almond Butter, Aloe Butter, Apricot Kernel Butter, Argan Butter , Attalea Maripa Seed Butter, Avocado Butter, Babassu Butter , Bacuri Butter, Bagura Soft Butter, Baobab Soft Butter, Bassia Butyracea Seed Butter, Bassia Latifolia Seed Butter, Black Currant Seed Butter, Brazil Nut Butter, Camelina Butter, Camellia Butter, Candelilla Butter , Carnauba Butter , Carpotroche Brasiliensis Seed Butter, Chamomile Butter, Cocoa Butter, Coconut Butter, Coffee Butter, Cotton Soft Butter, Cranberry Butter, Cupuacu Butter , Grape Seed Butter, Hazel Nut Butter, Hemp Seed Butter, Horsetail Butter, Illipe Butter, Irvingia Gabonensis Kernel Butter, Jojoba Butter, Karite Butter, Kokum Butter, Kukui Butter, Lavender Butter, Lemon Butter, Lime Butter, Macadamia Butter, Mango Butter, Marula Butter, Monoi Butter, Mowrah Butter , Mucaja Butter, Murumuru Butter, Olea Butter, Olive Butter, Orange Butter, Palm Oil, Passion Butter, Phulwara Butter, Pistachio Butter, Pomegranate Butter, Pumpkin Butter, Raspberry Butter, Rice Butter, Sal Butter, Sapucainha Butter, Seasame Butter, Shea Butter, Soy Butter Tamanu Butter, Sunflower Seed Butter, Sweet almond Butter, Tangerine Butter, Tucuma Seed Butter, Ucuuba Butter and Wheat Germ Butter.

Topical compositions of the invention may include surface active agents (surfactants). Surface-active agent refers to an ingredient that reduces surface tension and promotes skin cleansing. Many surface-active compounds also act as emulsifying agents, e.g., waxes, or foaming agents. There are four main categories of surface-active agents: cationic, anionic, amphoteric and nonionic. Amphoteric surface-active agents and nonionic surface-active agents are generally best tolerated by the epidermis. The surface-active agents C include, in particular, ammonium laureth sulphate; ammonium lauryl sulphate; caprylyl/capryl glucoside; cetyl betaine; cocamidopropyl betaine; coco-betaine; coco-glucoside; decyl glucoside; di sodium cocoamphodiacetate; disodium laureth sulphosuccinate; disodium lauryl sulphosuccinate; disodium stearoyl glutamate; glycol stearate; lauramidopropyl betaine; PEG-100 stearate; potassium cetyl phosphate; sodium cocoamphoacetate; sodium cocoyl isethionate; sodium laureth sulfate; sodium lauryl sulfate; sodium palm kernelate; sodium methyl cocoyl laurate; alpha olefin sulphonates such as sodium C₁₄-C₁₆ alpha olefin sulphonate; sodium lauroyl methyl isethionate; cocamidopropyl hydrosultaine; sodium lauroyl sarcosinate; sodium cocoyl glutamate; sodium cocoyl glycinate; sodium lauroyl lactylate; alkyl glucoside; alkyl polyglucoside; caprylic/capric glucoside lauryl ether; polysorbates such as polysorbate 80, polysorbate 20; sodium methyl sulpholaurate; sodium lauryl sulfoacetate; disodium sulfolaurate; soaps, which are fatty acid salts, of general formula RCOOM (R=long hydrocarbon chain with more than 10 carbon atoms, M=a metal, an alkali-metal or an organic base). Depending on the nature of the M group, there are alkali-metal soaps (Na⁺, K⁺, NH₄⁺ soaps), metal soaps (particularly of calcium) and organic soaps (for example, triethanolamine soap, including triethanolamine stearate) and combinations thereof. Advantageously, the surface-active agent is chosen among an anionic surface-active agent, an amphoteric surface-active agent, a nonionic surface-active agent and combinations thereof. Advantageously, the formulation comprises from 2 to 25% by weight, relative to its total weight, of surface-active agent.

Colorants, fragrances and abrasives may also be included in the delivery compositions. Each of these substances may range from 0.05 to 5%, such as from 0.1 and 3% by weight. Colorants of interest include titanium dioxide, where appropriate surface-treated (codified in the Color Index under the reference CI 77,891), manganese violet (CI 77,742), ultramarine blue (CI 77,007), chromium oxide (CI 77,288), hydrated chromium oxide (CI 77,289), ferric blue (CI 77,510), zinc oxide, zirconium dioxide. Specific colorants of interest include: D & C red no. 19 (CI 45,170), D & C red no. 9 (CI 15,585), D & C red no. 21 (CI 45,380), D & C orange no. 4 (CI 15,510), D & C orange no. 5 (CI 45,370), D & C red no. 27 (CI 45,410), D & C red no. 13 (CI 15,630), D & C red no. 7 (CI 15,850:1), D & C red no. 6 (CI 15,850:2), D & C yellow no. 5 (CI 19,140), D & C red no. 36 (CI 12,085), D & C orange no. 10 (CI 45,425), D & C yellow no. 6 (CI 15,985), D & C red no. 30 (CI 73,360), D & C red no. 3 (CI 45,430), carbon black (CI 77,266), cochineal carmine lake (CI 75,470), natural or synthetic melanin, and aluminium lakes.

Fragrances of interest include: Abies Alba Leaf Oil, Acetaldehyde, Acetanilid, Acetic Acid, Achillea Millefolium Oil, Actinidia Chinensis (Kiwi) Fruit Water, Adipic Acid, Agar, Alcohol Denat., Algin, Aloe Barbadensis Leaf , Amyl Acetate, Amyl Benzoate, Amyl Cinnamal, Anethole, Anise alcohol, Anthemis Nobilis Flower Water, Benzaldehyde, Benzyl Alcohol, Betula Alba Oil, Boswellia Serrata Oil, Butyl Acetate, Butyl Lactate, Calendula Officinalis Flower Oil, Camellia Sinensis Leaf Water, Camphor, Capsaicin, Cedrol, Cinnamal, Citral, Citronellol, Citrus Aurantifolia (Lime) Oil, Citrus Aurantium Dulcis (Orange) Oil, Citrus Grandis (Grapefruit) Oil, Citrus Tangerina (Tangerine) Peel Oil, Coumarin, Diacetone Alcohol, Ethyl Cinnamate, Ethyl Ether, Eucalyptus Caryophyllus (Clove) Flower Oil, Farnesol, Gardenia Florida Oil, Geranium Maculatum Oil, Hexyl Cinnamal, Hydrogenated Rosin, Illicium Verum (Anise) Oil, Isoamyl Acetate, Juniperus Mexicana Oil, Laurus Nobilis Oil, Lavandula angustifolia (Lavender) Oil, Melaleuca Alternifolia (Tea Tree) Leaf Oil, Melissa Officinalis Leaf Oil, Mentha Piperita (Peppermint) Oil, Menthol , 2-Naphthol, Origanum Majorana Leaf Oil, Panax Ginseng Root Extract, Pelargonic Acid, Pelargonim Graveolens Flower Oil, Pinus Silvestris Cone Oil, Prunus Armeniaca (Apricot) Kernel Oil, Rosa Canina Flower Oil, Rosmarinus Officinalis (Rosemary) Leaf Oil, Santalum Album (Sandalwood) Oil, Thymus Vugaris ( Thyme) Oil, Vanillin, Vitis Vinifera (Grape) Leaf Oil, Zingiber Officinale (Ginger) Root Oil.

In some instances, the compositions may include triclosan. This agent used in the formulation has been found effective against the whole genera of microorganisms, (for example: bacteria, fungi, Pseudomonas aeruginosa, Pseudomonas capacia, Staphylococcus aureus, Escherichia coli, Candida albicans, Aspergillus niger, Salmonella typhimurium, etc. . . . ).

Topical compositions of embodiments of the invention are free of antimicrobial amounts of disinfecting alcohols, e.g., ethyl or isopropyl alcohol. If an alcohol is present, it is present in amounts of 30% or less, such as 25% or less and including 20% or less. In some instances, the compositions are alcohol free. In some instances, the compositions may include an amount of a conditioning alcohol, such as cetyl alcohol, stearyl alcohol, cetearyl alcohol, etc.

### Utility

Topical formulations of the invention find use in a variety of applications. For examples, lotions and cleanser of the invention find use in applications where skin nourishment and disinfection are desired.

In practicing methods of the invention, a topical composition is applied to a topical region of a subject and maintained at the topical region for a period of time sufficient to result in the desired result, e.g., skin nourishment and/or cleansing/disinfection, such as mentioned above. The topical region is, in certain embodiments, a keratinized skin region. The keratinized skin region, including hair follicles, sweat glands and sebaceous glands, may be present at a variety of locations, e.g., limbs, arms, legs; torso, e.g., chest, back, stomach; head, e.g., neck, face; etc. In certain embodiments, the region will be a head region, such as a facial region, e.g., forehead, occipital region, around the mouth, etc. The topical region to which the composition is applied may vary with respect to area, ranging in certain embodiments from 1 mm² to 20,000 cm² or more, such as from 1 to 50 cm², and including from 3 to 10 cm².

Following application, the topical formulation is maintained at the site of application for a period of time sufficient for a desired therapeutic outcome to occur, e.g., amelioration of a symptom(s) of interest, reducing dryness. The period of time may vary, and in certain embodiments ranges from instantaneously up to several days, such as 10 seconds to 1 min to 24 hours or longer, such as from 30 min to 12 hours and including from 1 hour to 12 hours or longer.

Depending on the formulation, application may be followed by washing, e.g., where the topical composition is a cleanser.

The subject methods and compositions may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In certain embodiments, the subjects or patients are humans.

The subject topical formulations find use in applications where it is desired to nourish and/or clean/disinfect a topical location of a subject a subject.

Practice of methods of the invention can result in the improvement in skin, when there is a noticeable decrease in the amount of wrinkling, roughness, dryness, laxity, sallowness, or pigmentary mottling of the treated skin. Methods of measuring improvements in skin condition are well known in the art (see, e.g., Olsen et al., J. Amer. Acad. Dermatol. 26:215-24, 1992), and can include subjective evaluations by the patient or a second party, e.g., a treating physician. Objective methods can include skin topography measurements, such as those described in Grove et al., J. Amer. Acad. Dermatol. 21:631-37 (1989). In skin topography measurements, silicone rubber replicas are made of a small area of skin, e.g., a 1 cm diameter circular area. The silicone rubber replicas capture fine lines and wrinkles on the skin. These specimens are then analyzed using computerized digital image processing to provide an objective measurement of the skin's topography. Skin topography measurements generated following digital-image processing can be measured using the values Rₐ and R_{z} as described in Olsen et al., J. Amer. Acad. Dermatol. 37:217-26, 1997, where Rₐ represents the area of deviation of skin surface features above and below an average central line, and R_{z} represents the difference between the maximum and minimum heights in five equal segments of the skin surface profile. A statistically significant decline (e.g., P<0.05) in Rₐ and R_{z} values in skin treated according to the presence invention compared to untreated skin indicates an improvement in skin, as is achieved by practicing the methods of the invention.

The following examples are offered by way of illustration and not by way of limitation.

### EXPERIMENTAL

### I. Anti-Microbial Cleanser Compositions

### A. Formulation 1

### 1. Ranges

| Raw Materials | % w/w | Functions |
|---|---|---|
| Water | 60.0-70.0 | Solvent |
| Ammonium Laureth Sulfate | 10.0-25.0 | Surfactant |
| Lauramidopropyl Betaine | 5.0-10.0 | Surfactant |
| Hydroxyapatite | 0.1-5.0 | Skin condition/ |
| | | dermal delivery |
| Sorbitol | 1.0-5.0 | Humectant |
| Glycol Distearate | 1.0-5.0 | Emulsifier |
| Sodium Chloride | 0.1-2.0 | Stabilizer, |
| | | thickening |
| Chamomilla Recutita | 0.1-1.0 | Botanical, skin |
| (Matricaria) Flower Extract | | Conditioning |
| Hydroxyethylcellulose | 0.1-1.0 | Thickener |
| Triclosan | 0.1-1.0 | Preservative |
| Methylparaben | 0.1-0.5 | Preservative |
| Propylparaben | 0.1-0.5 | Preservative |
| Benzalkonium Chloride | 0.1-0.2 | Anti-microbial |

### B. Formulation 2

### 1. Ranges

| Raw Materials | % w/w | Function |
|---|---|---|
| Water | 60.0-70.0 | Solvent |
| Sodium Lauroyl Sarcosinate | 10.0-25.0 | Surfactant |
| Cocamidopropyl Betaine | 5.0-10.0 | Surfactant |
| Hydroxyapatite | 0.1-5.0 | Skin conditioning, |
| | | dermal delivery |
| Glycerin | 1.0-5.0 | Humectant |
| Glycol Stearate | 1.0-5.0 | Emulsifier |
| Cyamopsis Tetragonoloba | 0.1-1.0 | Thickener |
| (Guar) Gum | | |
| Aloe Barbadensis Leaf Juice | 0.1-1.0 | Botanical, skin |
| | | conditioning |
| Benzalkonium Chloride | 0.1-0.2 | Anti-microbial |
| Methylisothiazolinone/ | 0.02-0.1 | Preservative |
| Methylchloroisothiazolinone | | |

### B. Benefits

Frequent hand washing is the best way to protect against microbial infection but can lead to dry and damaged skin. The Anti-Microbial Cleansers reported above are liquid hand and body wash formulation designed for frequent use without drying effects. They are luxurious and smooth, packed with moisturizing nutrients and kill 99.9% of germs, leaving hands soft, and clean.

With continued daily use, visible improvements to skin condition can be expected within one week.

### Benefits:

Rapid new skin cell regeneration by accelerating barrier recovery
Stimulates fibroblast cells to produce elastin and collagen
Helps repair damaged skin
Improves stratum corneum integrity
Increases flexibility and skin rejuvenation
Reduces appearance of fine lines & wrinkles
soothing and healing for burns and small skin abrasions”

### Additional Product Features:

Fragrance-free
Hypoallergenic certified-safe for all skin types and ages
Natural, >95% vegetable-based
No animal by-products

### Additional Uses:

Prevents and eliminates protective glove irritation
Prevents and eliminates face mask irritation, “maskne”
Prevents and repairs disinfecting/antiseptic alcohol (e.g., isopropyl/ethyl alcohol)-damaged skin
Eliminate dermatitis, eczema and diaper rash
Eliminate body acne, and acne
Multi-purpose antiseptic and soothing-small burns, cuts and scrapes
Eliminates stubborn odors (feet & body)
Eliminate dry skin conditions, weathered, cracked or flakey skin
Anti-inflammatory
Antiseptic and healing: use for tattoos and skin abrasions
Every day non-irritating, non-drying sanitizer, which kills 99.9% microorganisms for hours, while improving the condition of your skin.

### II. Moisturizer

### A. Formulation 1

### 1. Ranges

| Raw Materials | % w/w | Function |
|---|---|---|
| Water | 70.0-80.0 | Solvent |
| Isopropyl Palmitate | 5.0-15.0 | Emollient |
| Emulsifying Wax | 3.0-10.0 | Emulsifier |
| Propanediol | 3.0-8.0 | Humectant |
| Hydroxyapatite | 0.1-5.0 | Skin conditioning, |
| | | dermal delivery |
| Sclerotium Gum | 0.1-2.0 | Thickener |
| Aloe Barbadensis Leaf Juice | 0.1-1.5 | Botanical, skin |
| | | conditioning |
| 1,2-Hexanediol & Caprylyl Glycol | 0.2-1.5 | Preservative |
| Tocopheryl Acetate | 0.1-1.0 | Vitamin E, |
| | | antioxidant |
| Benzalkonium Chloride | 0.1-0.2 | Anti-microbial |

### B. Formulation 2

### 1. Ranges

| Raw Materials | % w/w | Function |
|---|---|---|
| Water | 70.0-80.0 | Solvent |
| Di-PPG-3 Myristyl Ether Adipate | 5.0-15.0 | Emollient |
| Glyceryl Stearate & PEG-100 Stearate | 3.0-8.0 | Emulsifier |
| Butylene Glycol | 3.0-8.0 | Humectant |
| Hydroxyapatite | 0.1-5.0 | Skin conditioning, |
| | | dermal delivery |
| Polyacrylamide & C13-14 | 0.1-3.0 | Thickener, |
| Isoalkane & Laureth-7 | | stabilizer |
| Camellia Sinensis | 0.1-1.5 | Botanical, skin |
| (Green Tea) Leaf Extract | | conditioning |
| Tocopherol | 0.1-1.0 | Vitamin E, |
| | | antioxidant |
| Sodium Benzoate & Potassium Sorbate | 0.2-1.0 | Preservative |
| Benzalkonium Chloride | 0.1-0.2 | Anti-microbial |

### B. Benefits

The anti-Microbial Moisturizers described above kill 99.9% of microorganisms on skin and continue to protect against new germs for HOURS while simultaneously hydrating, nourishing and improving skin condition.

The above Anti-Microbial Moisturizers provide 4+ HOURS of sanitizing protection while delivery moisturizing nutrients into the skin allowing the nutrients to work from inside out to protect and improve your skin's health.
Proven effective for 4+ HOURS after application
Safely touch public surfaces with confidence
Antiseptic/disinfecting Alcohol-free and fragrance-free
Helps eliminate dry skin conditions
Certified Hypoallergenic; non-sensitizer
Safe for all ages and skin types; non-irritant
No animal by-products;

With continued daily use, visible improvements to skin condition can be expected within one week. Anti-microbial active ingredients kill 99.9% of microorganisms (Bacteria, Viruses, and Fungi) and remain on the skin surface, providing long-lasting sanitizing protection against new germs.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A topical composition comprising:
a non-triclosan antimicrobial agent;
a calcium phosphate component; and
a topical delivery vehicle.

2. The topical composition according to claim 1, wherein the antimicrobial agent comprises a quaternary ammonium compound.

3. The topical composition according to claim 2, wherein the quaternary ammonium compound is benzalkonium chloride.

4. The topical composition according to any of the preceding claims, wherein the calcium phosphate component comprises calcium phosphate particles.

5. The topical composition according to claim 4, wherein the calcium phosphate particles by the molecular formula Ca₁₀(PO₄)₆(OH)₂.

6. The topical composition according to any of the preceding claims, wherein the calcium phosphate component comprises hydroxyapatite.

7. The topical composition according to any of the preceding claims, wherein the composition further comprises at least one emollient.

8. The topical composition according to any of the preceding claims, wherein the composition further comprises one or more skin nourishing agents.

9. The topical composition according to any of the preceding claims, wherein the composition further comprises a preservative.

10. The topical composition according to claim 9, wherein the preseverative component comprises a paraben.

11. The topical composition according to claim 9, wherein the preservative component comprises triclosan.

12. The topical composition according to claim 9, wherein the preservative component is paraben free.

13. The topical composition according to any of the preceding claims, wherein the topical composition is a cleanser.

14. The topical composition according to claim 13, wherein the topical composition comprises a surfactant.

15. The topical composition according to any of claims 1 to 12, wherein the topical composition is a lotion.

16. A method comprising applying a topical composition according to any of the preceding claims to a skin surface of a subject.

17. The method according to claim 16, wherein the method is a method of cleaning the skin surface of the subject.

18. The method according to claim 16, wherein the method is a method of moisturizing the skin surface of the subject.

19. A method of making a topical composition according to any of claims 1 to 15.
